(19) United States
(12) Patent Application Publication
(10) Pub. No.: US 2007/0133738 A1
(43) Pub. Date: Jun. 14, 2007

(54) X-ray apparatus

(75) Inventor: Ulf Zimmermann, Aurachtal (DE)

Correspondence Address: SCHIFF HARDIN, LLP;PATENT DEPARTMENT, 6600 SEARS TOWER, CHICAGO, IL, 60606-6473, US

(73) Assignee: Siemens Aktiengeselischaft

(21) Appl. No.: 11/635,755

(22) Filed: Dec. 6, 2006

(30) Foreign Application Priority Data
Dec. 8, 2005 (DE) 10 2005 058 648.1

(51) Int. Cl. A61B 6/04

(52) U.S. Cl. 378/37

(57) ABSTRACT

An x-ray apparatus has an x-ray unit that is arranged on a stand and has a support arm on which an x-ray source and an x-ray detector are arranged. The x-ray unit can be positioned in at least one operating position at an angle laterally next to the stand, such that an access to the x-ray unit is possible from a front direction that corresponds to the viewing direction towards the support arm through an x-ray path, and from a rear direction opposite thereto, and from two further lateral directions that are orthogonal to the front direction and the rear direction.

### BACKGROUND OF THE INVENTION

1. Field of the Invention

The invention concerns an x-ray apparatus of the type used in mammography.

2. Description of the Prior Art

X-ray apparatuses as are used in medical imaging have an x-ray source and an x-ray detector. X-rays are directed from the x-ray source through a subject to be examined onto the x-ray detector. Upon the passage through the subject, x-rays are scattered and absorbed such that images of the inside of the subject can be produced from the radiation acquired by the x-ray detector.

A number of requirements are placed on x-ray apparatuses, in particular on x-ray apparatuses that are used for imaging relatively small body parts such as the breast or extremities.

The x-ray source and x-ray detector should be capable of being optimally freely positioned. For example, in mammography it is advantageous to irradiate a breast not only in the horizontal position of the breast but also to examine said breast at an angle or suspended vertically. Mammograms with subsequent biopsies, for example, are most simply effected on a recumbent patient with the breast suspended, since the force of gravity can be utilized for positioning the breast and the patient, when recumbent, can be examined more comfortably and with less movement. In contrast, other examinations can be implemented more simply upright with a horizontal position of the breast.

In addition to the movement capability of the x-ray source and the x-ray detector, a desire exists for a space-saving and mobile design in order to be able to also use the x-ray apparatus in small spaces and to be able to move it to another usage location in a simple manner. This requirement is in competition with the requirement for a mobile x-ray source and detector, since a relatively non-compact design must typically be utilized for the latter requirement.

For example, a mammography apparatus in which an x-ray source and an x-ray detector are arranged in a ring is known from EP 0 387 475 B1. The ring is supported such that it can rotate around its axis and around an axis perpendicular thereto and is mounted such that it can be displaced vertically on a stand. The x-ray source and the x-ray detector thus can be arranged in various spatial positions. The deployed annular design, however, occupies a relatively large amount of space.

From DE 101 63 236 A1 a mammography apparatus is known wherein an x-ray source and an x-ray detector are arranged on an examination arm that is mounted in a plane for an isocentric rotation. A support for the examination arm and a support column on which the support is mounted allow the height of this plane to be adjusted. Here as well the x-ray source and the x-ray detector can be arranged in various spatial positions corresponding to a standing, leaning, sitting or recumbent position of a patient. The design of the examination arm appears to be bulky.

A further requirement that is placed on such x-ray apparatuses is to have an optimally large access region to the body part to be examined. This allows body parts to be examined to be better positioned in the beam path, and causes distinctly less stress to the patient, who then can stand at the apparatus in an optimally free manner without bulky surrounding structures (as in the mammography apparatuses cited above), since such structure can produce an oppressive impression.

### SUMMARY OF THE INVENTION

An object of the present invention is to provide an x-ray apparatus in which a high degree of accessibility is ensured.

The object is inventively achieved by an x-ray apparatus according to the invention wherein an x-ray unit is arranged on a stand and has a support arm on which are arranged an x-ray source and an x-ray detector, the x-ray unit being positionable in at least one operating position at a lateral angle next to the stand, such that an access to the x-ray unit is permitted from a front direction that corresponds to the viewing direction toward the support arm through an x-ray path, and from a rear direction opposite thereto, and from two further directions that are orthogonal to the front direction and the rear direction.

In the inventive x-ray apparatus the components (the x-ray source and the x-ray detector) of the x-ray unit relevant for imaging are arranged relative to the stand such that the x-ray unit (and thus a body part to be examined) is freely accessible from four sides, namely from the front, the rear and from the sides. The four directions are established by the relative position of x-ray path and support arm. The front direction thereby corresponds to the viewing direction on the support arm through the x-ray path.

Because the x-ray unit is freely accessible from four sides, a user (for example an x-ray technician) will always find a possibility to approach the x-ray unit and the examination subject in a comfortable manner, such that a body part of a patient to be examined can be appropriately positioned in a suitable manner in the beam path. Even if the user approaches the x-ray unit from the rear, only the support arm and the compression unit (which can be easily accommodated by slender design) are located between the user and the beam path. A patient to be examined is not surrounded by bulky support structure that can produce an oppressive impression.

The x-ray unit preferably can rotate around a first axis that is parallel to the front direction and rear direction. The x-ray source and the x-ray detector can be arranged in various spatial positions in this manner, which enables acquisitions in different positions of the patient (standing, leaning, or recumbent).

The x-ray unit also can be made to rotate around a second axis that is orthogonal to the front direction and the rear direction. This axis likewise preferably proceeds orthogonal to the x-ray path. The positioning possibilities of the x-ray source and the x-ray detector are thereby increased.

In addition to the at least one operating position, the x-ray unit preferably can be positioned in a transport position laterally next to the stand, such that the x-ray unit is protected by the stand on one side. In this space-saving transport position the x-ray apparatus can be moved from one usage location to another. Because the x-ray unit is protected on one side by the stand, impacts with objects can be better avoided than with a freely accessible x-ray unit. Moreover, the center of gravity lies lower, such that the transport can be performed more safely.

In an embodiment, the x-ray unit is arranged on the stand such that it is adjustable in terms of height. The positioning possibilities of the x-ray source and of the x-ray detector are thereby increased. The x-ray unit can be adjusted to the size or the position of the patient—sitting, recumbent or standing.

In another embodiment, the stand on which the x-ray unit is arranged is itself adjustable in terms of height. In this embodiment as well the x-ray unit can be adapted to the size or the position of the patient.

In a further embodiment of the x-ray apparatus, the x-ray unit is arranged on the stand via a pivotable arm. The movement possibilities of the x-ray unit are increased with this design.

The x-ray apparatus preferably is fashioned such that it is suitable for use for mammography since an optimally free access to the x-ray unit is particularly necessary (in order to position a breast to be examined) together with a high movement capability of the x-ray unit.

### DESCRIPTION OF THE DRAWINGS

FIG. 1, FIG. 2 and FIG. 3 show a lateral view, a front view and a plan view, respectively, of an x-ray apparatus in an operating position in which an x-ray unit is arranged at an angle laterally next to a stand in accordance with the invention.

FIG. 4, FIG. 5 and FIG. 6 show various operating positions of the x-ray unit in a lateral view, a front view, and a plan view, respectively.

FIG. 7 shows a transport position of the x-ray unit in addition to the stand.

FIG. 8 shows a special design of the x-ray unit with further positioning possibilities.

FIG. 9 and FIG. 10 show a lateral view and a front view, respectively, of an x-ray apparatus in which an x-ray unit is arranged on a pivotable arm.

### DESCRIPTION OF THE PREFERRED EMBODIMENTS

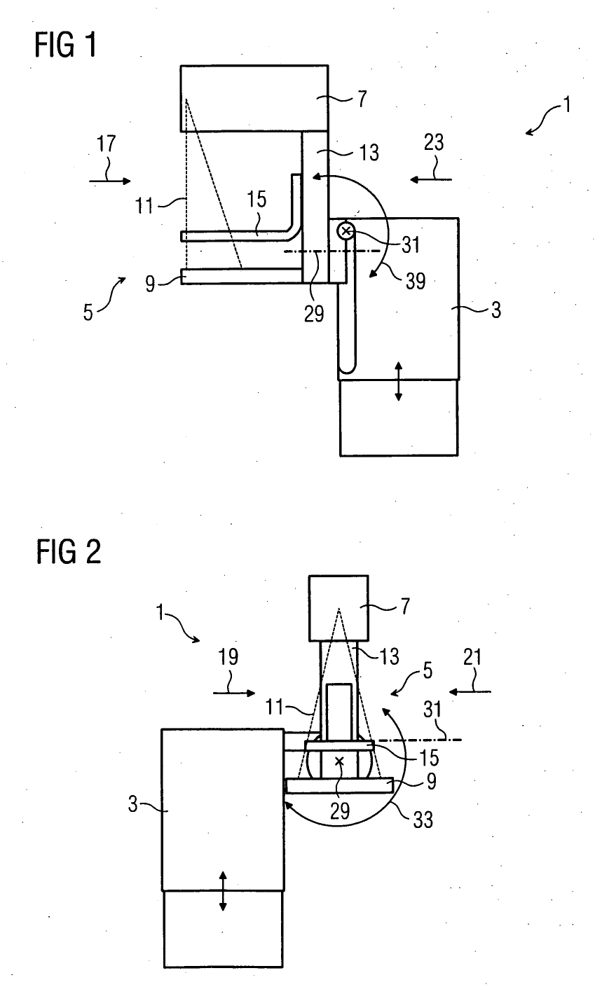

FIG. 1 through FIG. 3 respectively show a lateral view, a front view and a plan view of an x-ray apparatus 1 that has a stand 3 and an x-ray unit 5. In an operating position of the x-ray unit 5 the x-ray unit 5 is arranged at an angle laterally next to the stand 3. The x-ray unit 5 has an x-ray source 7 from which x-ray radiation 11 is emitted and an x-ray detector 9 that acquires the x-ray radiation 11. The x-ray source 7 and the x-ray detector 9 are arranged on a support arm 13.

If the x-ray apparatus 1 is fashioned for mammography (as shown here), a compression plate 15 with which a breast to be examined can be compressed is located in the beam path of the x-ray radiation 11.

As in known mammography apparatuses, the x-ray unit 4 can be accessed from a front direction 17. The front direction 17 essentially corresponds to the viewing direction of the support arm 13 through the beam path of the x-ray radiation 11. This is also the direction from which typical patients approach the x-ray unit 5. The x-ray unit 5 is furthermore accessible from two lateral directions 19, 21 that are orthogonal to the front direction 17.

Because the x-ray unit 5 is inventively arranged at an angle laterally next to the stand 3, a free access to the x-ray unit 5 is possible even from a rear direction 23 that is precisely opposite to the front direction 17.

As shown in FIG. 3, a patient 27 can step up to the x-ray unit 5 from, for example, the front direction 17 for an acquisition. Due to the particular mounting of the x-ray unit 5 on the stand 3, an assistant 25 can step up to the x-ray unit 4 from a number of directions. It is now also possible for an assistant 25 to approach the x-ray unit 5 from a rear direction 23. This direction is therefore advantageous since the assistant 25 stands opposite the patient 27. For example, the assistant 25 can then more easily position the breast to be examined in the beam path in that the assistant 25 can exert a direct pull on the breast to pull it forward toward the assistant 25 with a suitably narrow embodiment of the support arm 13, this is possible without further measures.

During the positioning of the breast the assistant 25 can additionally keep direct eye contact with the patient 27, which for many patients 27 brings an important alleviation of the examination situation that is felt to be burdensome.

The attachment of the x-ray unit 5 on the stand 3 ensues via an articulation that enables a rotation of the x-ray unit 5 around two axes 29, 31. The first axis 29 is essentially parallel to the front direction 17. This allows the x-ray unit 5 to be rotated corresponding to the first double arrow 33. Furthermore, a rotation is possible around a second axis 31 (corresponding to the second double arrow 39) that is orthogonal to the first axis 29 and to the direction of the x-ray radiation 11. The movement possibilities of the x-ray unit 5 are also increased by this feature. The x-ray unit 5 can be rotated around both axes 29, 31 over an angle range of 360°.

In the exemplary embodiment shown here, the stand 3 is designed as a height-adjustable stand 3 such that the height of the x-ray unit 5 can be regulated via this. The height of the x-ray unit 5 can furthermore additionally be regulated in that the x-ray unit 5 itself can be adjusted in terms of its height on the stand 3. The x-ray detector can advantageously be displaced across a height range of at least 650 mm to 1450 mm.

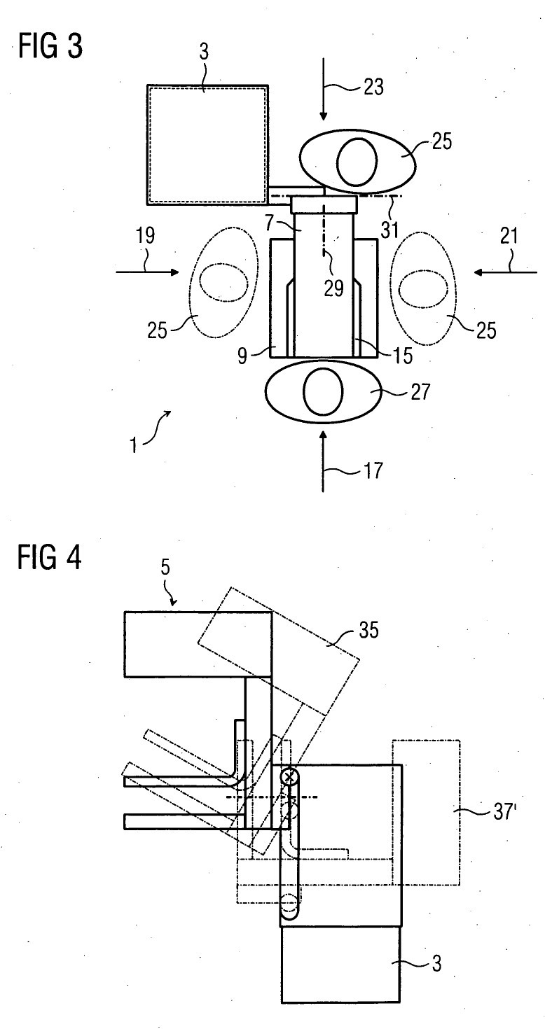

Due to the rotation possibilities of the x-ray unit 5 it can be operated in various operating positions that are now described in FIG. 4 through FIG. 6. For example, an acquisition of the breast in a state suspended at an angle can be produced via various angled operating positions 35. The height and the angle of inclination are thereby adapted to the necessities of the required examination and the size of the patient 27 to be examined.

When the x-ray unit 5 is tilted by 90° around the first or second axis 29, 31, horizontal operating positions 37, 37′ result that are particularly suitable for a recumbent examination position with a vertically-depending breast. A patient is thereby moved over the x-ray unit 5 with a bed (not shown here) that has a recess at a corresponding position. The height of the stand 3 and of the x-ray unit 5 are correspondingly adapted to the height of the bed.

The horizontal operating position 37′ at which the x-ray unit has been rotated by 90° around the second axis 31 is particularly suited for the production of exposures in the recumbent position since then the position of the breast relative to the x-ray unit 5 is the same as from a front direction 17 given an acquisition in the standing position when the patient 27 stands in front of the x-ray unit 5, as is shown in FIG. 3. In this operating position 37′ the support arm 13 is located below such that, for a possible biopsy that is possibly taken in the same examination course, sufficient space remains present from the side in order to position biopsy devices (not shown here).

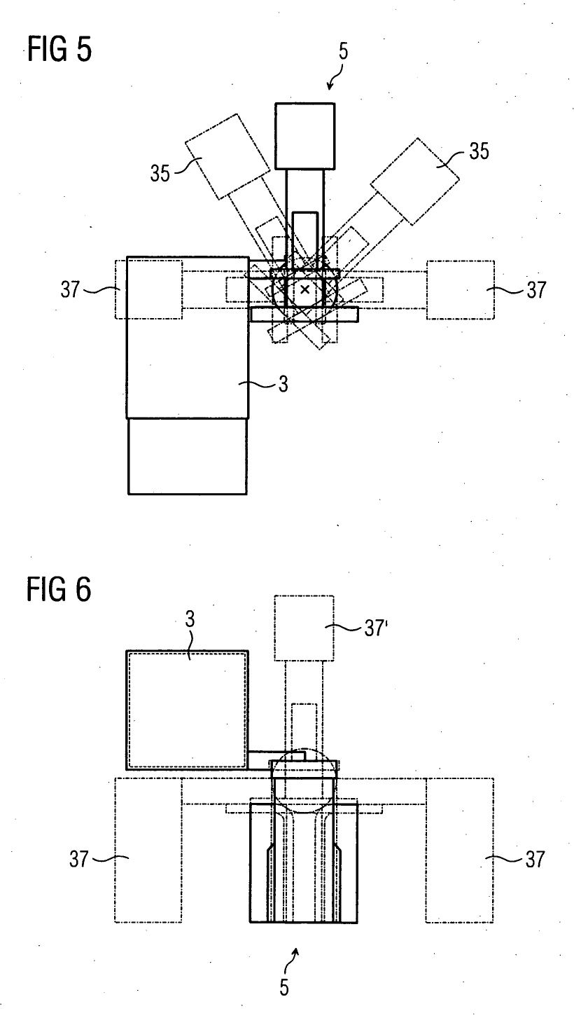

FIG. 7 shows a transport position 49 of the x-ray unit 5. Via a rotation by 180° around the second axis 31 the x-ray unit 5 can be positioned such that the x-ray unit 5 is located at the same height next to the stand 3. This position is particularly suited for a transport since here the x-ray apparatus 1 takes up less space. The x-ray unit 5 is additionally protected from impacts or the like on one side by the tripod 3.

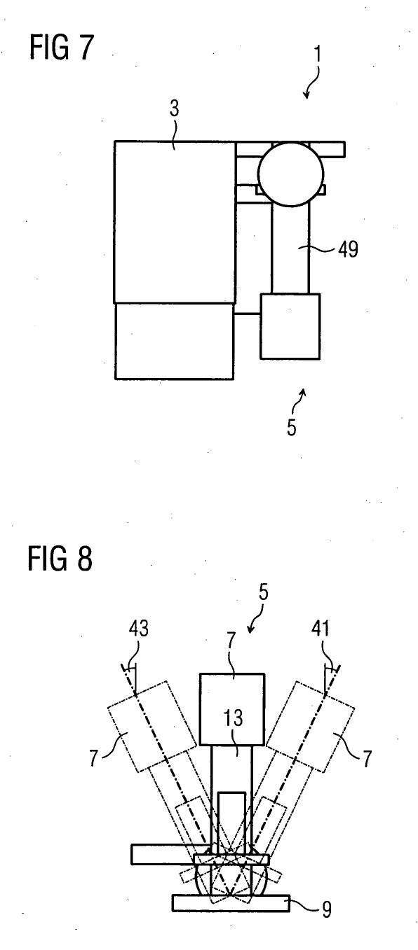

FIG. 8 shows a special design of the support arm 13 that can be adjusted here such that the x-ray radiation 11 can be directed onto the breast from various angles 41, 43. The x-ray source 7 is thereby only tilted while the x-ray detector 9 retains its position. The angle range thereby typically extends up to ±25°. Tomographies and stereotactic examinations can be effected with a support arm 13 fashioned in such a manner.

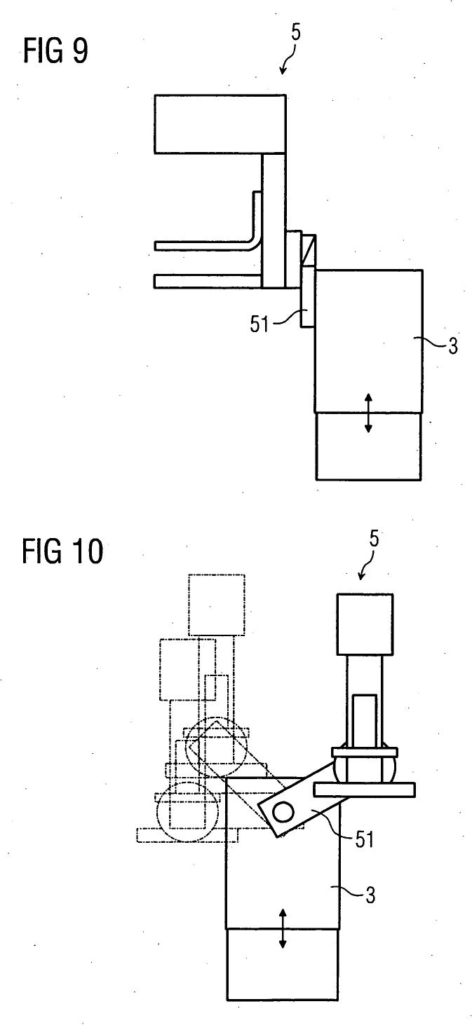

FIG. 9 and FIG. 10 respectively show a side view and a front view of an x-ray apparatus 1 in which an x-ray unit is arranged on a stand 3 via a pivotable arm 51. The movement possibilities of the x-ray unit 5 are further increased.

Although modifications and changes may be suggested by those skilled in the art, it is the intention of the inventors to embody within the patent warranted hereon all changes and modifications as reasonably and properly come within the scope of their contribution to the art.

I claim as my invention:

1. An x-ray apparatus comprising:
a support stand;
an x-ray unit mounted on said support stand, said x-ray unit comprising a support arm and an x-ray source and an x-ray detector mounted on said support arm; and
an attachment arrangement that attaches said x-ray unit to said support stand with said x-ray unit disposed at an angle laterally next to said stand, and allowing access to said x-ray unit from a front direction corresponding to a viewing direction toward the support arm through a beam path of x-rays emitted by said x-ray source, and from a rear direction opposite to said front direction, and from two further lateral directions that are orthogonal to said front direction and said rear direction.

2. An x-ray apparatus as claimed in claim 1 wherein said attachment arrangement allows said x-ray unit to be rotated around a first axis parallel to said front direction and said rear direction.

3. An x-ray apparatus as claimed in claim 2 wherein said attachment arrangement allows said x-ray unit to be rotated around a second axis that is orthogonal to said front direction and said rear direction.

4. An x-ray apparatus as claimed in claim 3 wherein said second axis is orthogonal to said direction of said x-ray beam.

5. An x-ray apparatus as claimed in claim 1 wherein said attachment arrangement allows said x-ray unit to be positioned in a transport direction laterally next to said support stand, with said support stand protecting said x-ray unit at one side of said x-ray unit.

6. An x-ray apparatus as claimed in claim 1 wherein said attachment arrangement allows height-adjustment of said x-ray unit relative to said support stand.

7. An x-ray apparatus as claimed in claim 1 wherein said support stand is height-adjustable.

8. An x-ray apparatus as claimed in claim 1 wherein said attachment arrangement comprises a pivotable arm connecting said x-ray unit to said support stand.

9. An x-ray apparatus as claimed in claim 1 wherein said x-ray unit comprises a compression plate for mammography examinations.
